(19) United States
(12) Patent Application Publication
(10) Pub. No.: US 2024/0285933 A1
(43) Pub. Date: Aug. 29, 2024

(54) CANNULA WITH DIP COATED INNER POLYMER

(71) Applicant: Boston Scientific Scimed, Inc., Maple Grove, MN (US)

(72) Inventors: Qian Liu, Plymouth, MN (US); Reggie Roth, Monticello, MN (US)

(73) Assignee: Boston Scientific Scimed, Inc., Maple Grove, MN (US)

(21) Appl. No.: 18/583,201

(22) Filed: Feb. 21, 2024

Related U.S. Application Data
(60) Provisional application No. 63/447,715, filed on Feb. 23, 2023.

(51) Int. Cl. A61M 60/857; A61M 25/00; A61M 60/174; A61M 60/216

(52) U.S. Cl. CPC A61M 60/857; A61M 60/174; A61M 60/216; A61M 25/0013; A61M 25/0045; A61M 25/005; A61M 2205/0266; A61M 2207/10

(57) ABSTRACT

A method for coating a cannula having a proximal portion opposite a distal portion and a cannula body extending between the distal portion and the proximal portion includes, providing the cannula body, loading the cannula body onto a mandrel, dipping the cannula body into a polymer solution to form a dip coating along an inner surface of the cannula body, and applying an outer layer of polymer onto a cannula.

### CROSS-REFERENCE TO RELATED APPLICATIONS

This application claims the benefit of U.S. Provisional Patent Application Ser. No. 63/447,715, filed Feb. 23, 2023, which is incorporated herein by reference.

### TECHNICAL FIELD

The present disclosure relates to percutaneous circulatory support devices. More specifically, the disclosure relates to percutaneous circulatory support devices coupled with a cannula.

### BACKGROUND

Circulatory support devices support the pumping action of the heart. These devices may be disposed through a valve opening such as, for example, an aortic valve. Blood flow through the circulatory support devices is an important factor when differentiating between different types of circulatory support devices. In some instances, a cannula may be used for providing blood flow to the circulatory support device. Proper coating of the cannula is required for allowing sufficient blood flow through the cannula and to the circulatory support device.

### SUMMARY

In Example 1, a method for coating a cannula having a proximal portion opposite a distal portion and a cannula body extending between the distal portion and the proximal portion includes, providing the cannula body, loading the cannula body onto a mandrel, dipping the cannula body into a polymer solution to form a dip coating along an inner surface of the cannula body, and applying an outer layer of polymer onto a cannula.
In Example 2, the method of Example 1 further includes wherein the cannula body of the cannula is a braided structure composed of nitinol.
In Example 3, the method of Example 1 further includes wherein the cannula body of the cannula is a laser cut tube of nitinol or a coil structure composed of nitinol.
In Example 4, the method of any one of Examples 1-3, further includes wherein the polymer solution is composed of a thermoset polymer.
In Example 5, the method of any one of Examples 1-4, further includes wherein the outer layer of polymer applied to the cannula is a layer of a thermoset polymer.
In Example 6, the method of any one of Examples 1-5, further includes wherein dipping the cannula body into the polymer solution includes rotating the mandrel and the cannula body such that the polymer solution is disposed evenly along the inner surface of the cannula body.
In Example 7, the method of Example 6 further includes wherein the mandrel is rotated at a speed of between approximately 5 rpm and approximately 50 rpm.
In Example 8, the method of any one of Examples 1-7 further includes wherein the outer layer defines an outer diameter.
In Example 9, the method of Example 8 further includes wherein the outer diameter has a value ranging between approximately 0.1 inches and approximately 0.3 inches.
In Example 10, the method of any one of Examples 1-9 further includes wherein the dip coating defines an inner layer, and the inner layer defines an inner diameter of the cannula.
In Example 11, the method of Example 10 further includes wherein the inner diameter has a value ranging between approximately 0.09 inches and approximately 0.29 inches.
In Example 12, a method of coating a cannula having a proximal portion opposite a distal portion and a cannula body extending between the distal portion and the proximal portion includes providing the cannula body, loading the cannula body onto a mandrel, dipping the cannula body into a polymer solution to form an inner layer of coating along a surface of a lumen of the cannula body, attaching an adhesive layer onto an outer surface of the cannula body, and applying an outer layer of polymer onto the cannula.
In Example 13, the method of Example 12 further includes wherein the inner layer defines an inner diameter of the cannula and the outer layer of the polymer defines an outer diameter of the cannula.
In Example 14, the method of Example 13 further includes wherein the inner diameter has a value of between approximately 0.09 inches and approximately 0.29 inches.
In Example 15, the method of Example 13 or Example 14 further includes wherein the outer diameter has a value of between approximately 0.1 inches and approximately 0.3 inches.
In Example 16, a method of coating a cannula for use with a circulatory support device including a proximal portion opposite a distal portion and a cannula body extending between the distal portion and the proximal portion, the cannula body defined by an inner surface and an outer surface, including providing the cannula body, loading the cannula body onto a mandrel, dipping the cannula body into a polymer solution to form a dip coating along the inner surface of the cannula body, and applying an outer layer of polymer onto the cannula.
In Example 17, the method of Example 16 further includes wherein the cannula body is a braided structure of nitinol.
In Example 18, the method of Example 16 further includes wherein the cannula body is a laser cut tube of nitinol or a coil structure composed of nitinol.
In Example 19, the method of Example 16 further includes wherein the polymer solution is composed of a thermoset polymer.
In Example 20, the method of Example 16 further includes wherein the outer layer of polymer applied to the cannula is a layer of a thermoset polymer.
In Example 21, the method of Example 16 further includes wherein dipping the cannula body into the polymer solution includes rotating the mandrel and the cannula body such that the polymer solution is disposed evenly along the inner surface of the cannula body.
In Example 22, the method of claim 21 further includes wherein the mandrel is rotated at a speed of between approximately 5 rpm and approximately 50 rpm.
In Example 23, the method of Example 16 further includes wherein the outer layer defines an outer diameter.
In Example 24, the method of Example 23 further includes wherein the outer diameter has a value ranging between approximately 0.1 inches and approximately 0.3 inches.
In Example 25, the method of Example 16 further includes wherein the dip coating defines an inner layer and the inner layer defines an inner diameter of the cannula.
In Example 26, the method of Example 25 further includes wherein the inner diameter has a value ranging between approximately 0.09 inches and approximately 0.29 inches.
In Example 27, the method of Example 16 further includes wherein prior to applying the outer layer onto the cannula, the method further includes attaching an adhesive layer to the cannula body.
In Example 28, the method of Example 27 further includes wherein applying the outer layer onto the cannula includes attaching the outer layer to the adhesive layer.
In Example 29, a method of coating a cannula for use with a circulatory support device having a proximal portion opposite a distal portion and a cannula body extending between the distal portion and the proximal portion includes providing the cannula body, loading the cannula body onto a mandrel, dipping the cannula body into a polymer solution to form an inner layer of coating along a surface of a lumen of the cannula body, attaching an adhesive layer onto an outer surface of the cannula body, and applying an outer layer of polymer onto the cannula.
In Example 30, the method of Example 29 further includes wherein the inner layer defines an inner diameter of the cannula and the outer layer of the polymer defines an outer diameter of the cannula.
In Example 31, the method of Example 30 further includes wherein the inner diameter has a value of between approximately 0.09 inches and approximately 0.29 inches.
In Example 32, the method of Example 30 further includes wherein the outer diameter has a value of between approximately 0.1 inches and approximately 0.3 inches.
In Example 33, the method of Example 29 further includes wherein the polymer solution is composed of polyurethane or silicone.
In Example 34, the method of Example 29 further includes wherein the polymer of the outer layer is composed of polyurethane or silicone.
In Example 35, the method of Example 29 further includes wherein dipping the cannula body into the polymer solution includes rotating the mandrel and the cannula body such that the polymer solution is disposed evenly along the inner surface of the cannula body.
In Example 36, the method of any of the preceding Examples, wherein the dip coating is also formed along an outer surface of the cannula.
In Example 37, the method of Example 36, wherein the dip coating on the outer surface is at least partially covered by the outer layer.
In Example 38, the method of Example 36, wherein the dip coating on the outer surface is completely covered by the outer layer.

### BRIEF DESCRIPTION OF THE DRAWINGS

FIG. 1 illustrates a conceptual diagram of a circulatory support device including a cannula, in accordance with embodiments of the subject matter disclosed herein.

FIG. 2 is a side sectional view of several components of an illustrative percutaneous circulatory support device, in accordance with embodiments of the subject matter disclosed herein.

FIG. 3 is a side view of a cannula of the illustrative percutaneous circulatory support device of FIG. 2, in accordance with embodiments of the subject matter disclosed herein.

FIG. 4 is a side view of a cannula of the illustrative percutaneous circulatory support device of FIG. 2, in accordance with embodiments of the subject matter disclosed herein.

FIG. 5 is a cross-sectional view of the cannula of FIG. 3, in accordance with embodiments of the subject matter disclosed herein.

FIG. 6 is a flow chart of a method of coating a cannula, in accordance with embodiments of the subject matter disclosed herein.

### DETAILED DESCRIPTION

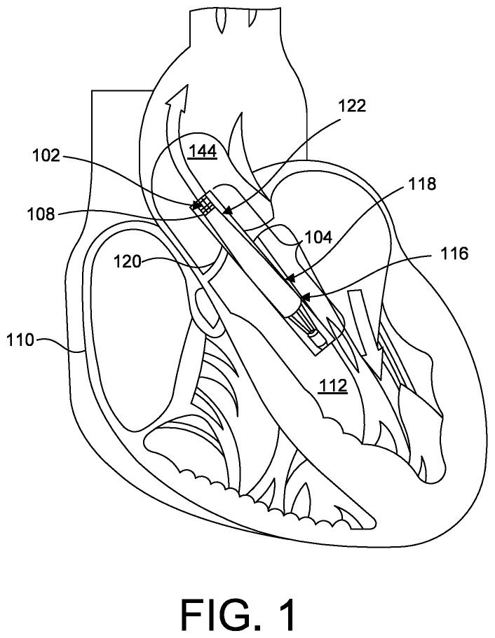

FIG. 1 depicts a conceptual diagram of a circulatory support device 102 coupled with a cannula 104, in accordance with embodiments of the subject matter disclosed herein. The cannula 104 and circulatory support device 102 are shown arranged within a heart 110 and aorta 114. According to embodiments, the circulatory support device 102 (also referred to herein, interchangeably, as a “blood pump”) is coupled to the cannula 104 by the connector 108. As shown in FIG. 1, the circulatory support device 102 and cannula 114 are configured to pump blood from the subject's left ventricle 112 into the subject's aorta 114. In embodiments, the circulatory support device 102 may be used to treat cardiogenic shock and other heart failure modalities.

In the embodiment of FIG. 1, a distal portion 116 of the cannula 104 is arranged in the left ventricle 112. An intermediate portion 118 of the cannula 104 extends through the aortic valve 120 so that a proximal portion 122 of the cannula 104 extends into the aorta 114. In embodiments, the proximal portion 122 of the cannula 104 is coupled to the connector 108 and the connector 108 is coupled to the circulatory support device 102. In other embodiments, the cannula 104 is coupled to the circulatory support device 102 without the use of a connector. During operation, the circulatory support device 102 draws blood from the left ventricle 112, through the cannula 104 and the circulatory support device 102, and then blood is released into the aorta 114. Additionally, or alternatively, the circulatory support device 102 may be used to facilitate pumping blood from some other aspect of the subject's vasculature into an adjacent portion of the vasculature.

FIG. 1 is not intended to suggest any limitation as to the scope of use or functionality of embodiments of the present disclosure. FIG. 1 also should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the present disclosure.

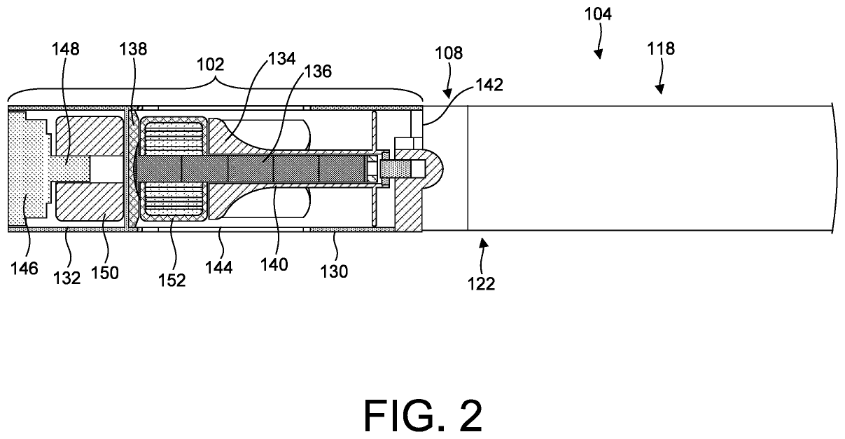

FIG. 2 depicts a partial side sectional view of the circulatory support device 102 depicted in FIG. 1 and the cannula 104, in accordance with embodiments of the subject matter disclosed herein. As previously disclosed with reference to FIG. 1, the cannula 104 may include a proximal portion 122, an intermediate portion 118, and a distal portion 116. In embodiments, the proximal portion 122 is positioned coupled to the connector 108 which is then operatively coupled to the remainder of the circulatory support device 102, the components of which will be described further herein.

With continued reference to FIG. 2, the circulatory support device 102 generally includes an impeller housing 130 and a motor housing 132. In some embodiments, the impeller housing 130 and the motor housing 132 may be integrally or monolithically constructed. In other embodiments, the impeller housing 130 and the motor housing 132 may be separate components configured to be removably or permanently coupled.

The impeller housing 130 carries an impeller assembly 134 therein. The impeller assembly 134 includes an impeller shaft 136 that is rotatably supported by at least one bearing, such as a bearing 138. The impeller assembly 134 also includes an impeller 140 that rotates relative to the impeller housing 130 to drive blood through the device 102. More specifically, the impeller 140 causes blood to flow from a blood inlet 142 formed on the impeller housing 130, through the impeller housing 130, and out of a blood outlet 144 formed on the impeller housing 130. In some embodiments and as illustrated, the impeller shaft 136 and the impeller 140 may be separate components, and in other embodiments the impeller shaft 136 and the impeller 140 may be integrally formed. In some embodiments and as illustrated, the inlet 142 and/or the outlet 144 may each include multiple apertures. In other embodiments, the inlet 142 and/or the outlet 144 may each include a single aperture. In some embodiments and as illustrated, the inlet 142 may be formed on an end portion of the impeller housing 130 and the outlet 144 may be formed on a side portion of the impeller housing 130. In other embodiments, the inlet 142 and/or the outlet 144 may be formed on other portions of the impeller housing 130. As illustrated and previously described, the impeller housing 130 couples to the cannula 104 such that the cannula 104 receives and delivers blood to the blood inlet 142.

With continued reference to FIG. 2, the motor housing 132 carries a motor 146, and the motor 146 is configured to rotatably drive the impeller 140 relative to the impeller housing 130. In the illustrated embodiment, the motor 146 rotates a drive shaft 148, which is coupled to a driving magnet 150. Rotation of the driving magnet 150 causes rotation of a driven magnet 152. The driven magnet 152 is connected to and rotates together with the impeller assembly 134. More specifically, in embodiments incorporating the impeller shaft 136, the impeller shaft 136 and the impeller 140 are configured to rotate with the driven magnet 152. In other embodiments, the motor 146 may couple to the impeller assembly 134 via other components. Further, in some embodiments, the motor 146 may be arranged outside of the circulatory support device 102 and is electrically coupled with the circulatory support device 102 for driving the impeller 140.

In some embodiments, a controller (not shown) may be operably coupled to the motor 146 and configured to control the motor 146. In some embodiments, the controller may be disposed within the motor housing 132. In other embodiments, the controller may be disposed outside of the motor housing 132 (for example, in a catheter handle, an independent housing, etc.). However, the above described embodiment of the circulatory support device 102 is not meant to be limiting and the cannula 104, and any variations of the cannula described herein with reference to FIGS. 1-5, may be used with variations of other circulatory support devices. Even further, the cannula 104 described herein may be used with various other percutaneous devices. The cannula 104, and various embodiments thereof, will be described further herein.

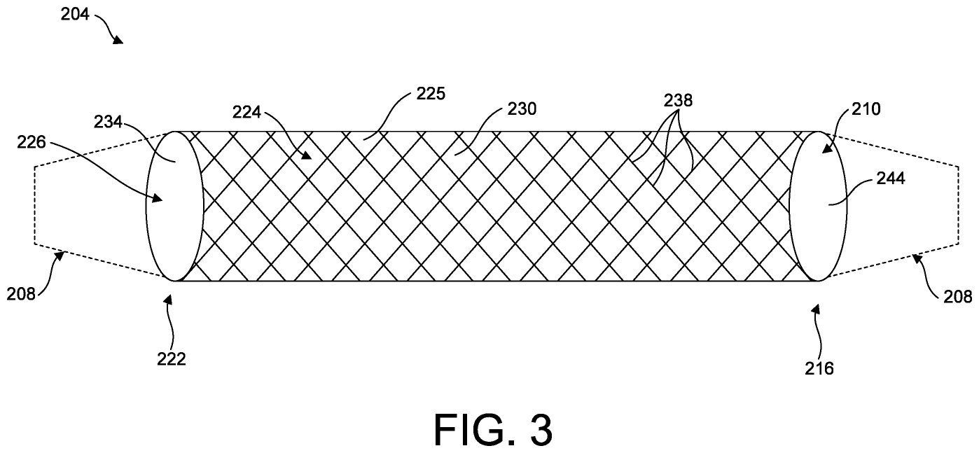

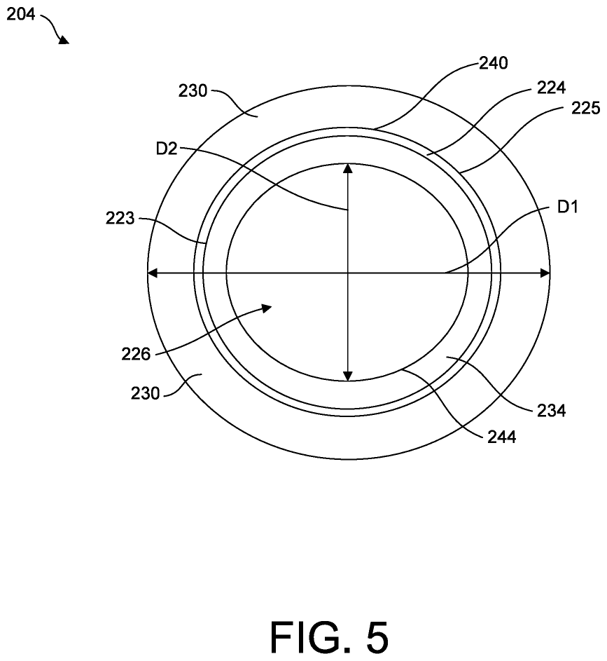

FIG. 3 illustrates an embodiment of a cannula 204 that may be used in combination with the circulatory support device 102 of FIG. 1. Similar to the cannula 104 of FIG. 1, the cannula 204 comprises a proximal portion 222, an intermediate portion 218, and a distal portion 216. The cannula 204 comprises a cannula body 224 and a lumen 226 extending therethrough. The cannula body 224 may have an inner surface 223 (FIG. 5) and an outer surface 225. As described in more detail below, the cannula 204 also comprises an inner layer 234 and outer layer 230. The lumen 226 is configured for the passage of blood into the cannula 204 and into the blood inlet 142 (FIG. 2) of the device 102 (FIG. 1). The impeller 140 may cause blood to flow into a cannula inlet 210, through the cannula lumen 226, and into blood inlet 142. In other embodiments, the impeller 140 may cause blood to flow into one or more openings arranged within the cannula body 224, through the cannula lumen 226, and into the blood inlet 142. However, in other embodiments, the cannula 204 may include several other openings or mechanisms for receiving the blood flow caused by impeller 140. The components of the cannula 204 may be formed from a variety of materials. For example, the cannula body 224 may be composed of materials including, but not limited to, nitinol, stainless steel, Inconel, and MP35N.

Further, with continued reference to FIG. 3, the cannula body 224 is defined by a braided structure 238. In some embodiments, the braided structure 238 may be composed of nitinol, however, various other materials may be incorporated. Further, in some embodiments, the cannula body 224 may be defined by a coiled structure formed of nitinol. In other embodiments, the cannula body 224 may be defined by various other structures formed of nitinol, for example laser cut nitinol.

The cannula 204 additionally includes the outer layer 230, which may be disposed on the outer surface 225 of the cannula body 224. The outer layer 230 may be formed of a variety of materials, including but not limited to, polyurethane, silicone, HYTREL®, polyether block amide, or various other thermoformed or thermoset polymers. More particularly, the outer layer 230 may be a polymer sheet formed of one of the above noted materials that is disposed around the cannula body 224 and extends from the proximal portion 222 to the distal portion 216. The outer layer 230 may be coupled, adhered, or otherwise attached to the outer surface 225 of the cannula body 224 of the cannula 204.

The cannula 204 additionally includes the inner layer 234, or a dip coating layer, which may be composed of a polymer such as polyurethane or silicone, formed through dip coating the cannula 204, as will be described further herein.

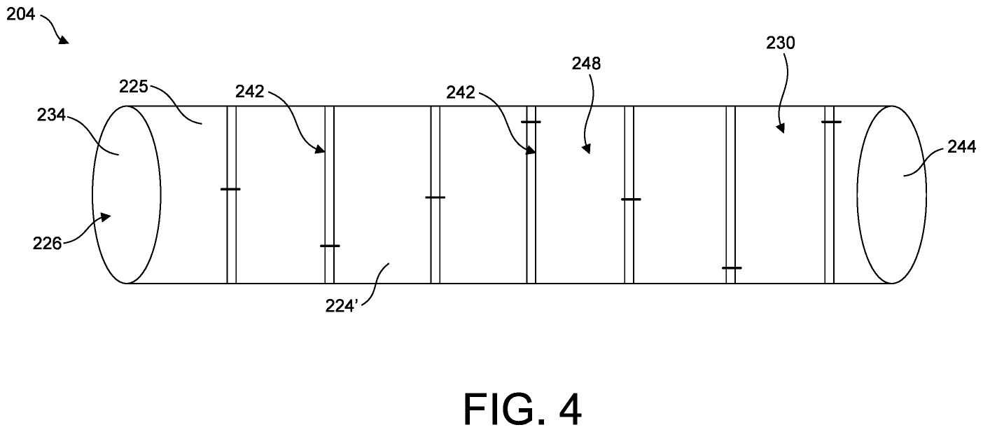

While the cannula body 224 of the cannula 204 is illustrated in FIG. 3 as being the braided structure 238, various other forms of the cannula body 224 may be incorporated. For example, FIG. 4 illustrates an additional embodiment of the cannula 204 having a modified cannula body 224′. For example, the cannula body 224′ is composed of a laser cut tube 248. In these instances, the laser cut tube 248 may be a tube formed of nitinol, or another applicable material, that is laser cut such that it has a plurality of slots 242 disposed within the cannula body 224′.

With reference now to the cross-sectional view of FIG. 5, the various layers of the cannula 204 will be described further herein. While the disclosure herein is described with reference to the cannula 204 having the cannula body 224 as shown in FIG. 3, the description may also be applied to the cannula 204 having the modified cannula body 224′ as shown in FIG. 4. FIG. 5 illustrates the cannula body 224 having the outer layer 230 disposed around the outer surface 225 of the cannula body 224. The outer layer 230 defines an outer diameter D1 of the cannula 204. The value of the outer diameter D1 may range from approximately 0.1 inches (0.254 cm) and approximately 0.3 inches (0.762 cm). As illustrated, the outer layer 230 is disposed about the cannula 204 such that the outer layer 230 defines a smooth outer surface. In some embodiments, and as illustrated in FIG. 5, the cannula 204 includes an adhesive layer 240 that is disposed onto the outer surface 225 of the cannula body 224 of the cannula 204 and is arranged between the outer surface 225 and the outer layer 230. In this way, the adhesive layer 240 may be coupled or otherwise attached to the cannula body 224, and more specifically to the outer surface 225, prior to the attachment of the outer layer 230 to the adhesive layer 240 of the cannula body 224. In these instances, the adhesive layer 240 may be configured for increasing the strength of the attachment of the outer layer 230 to the cannula body 224 of the cannula 204. In some embodiments, the adhesive layer 240 may be composed of DYMAX® 204-CTH, DYMAX® 203A-CTH-F, or various other applicable materials. In further embodiments, the outer layer 230 may be attached to the cannula body 224 through welding of the outer layer 230 and the cannula body 224 with adapters arranged on either end of the cannula 204. As will be described further herein, the adaptors may aid in attachment of the cannula 204 to a mandrel, among various other purposes.

Further, the cannula 204 includes the inner layer 234 disposed on the inner surface 223 of cannula body 224 and defining the lumen 226 of the cannula 204. The inner layer 234 may also be arranged on or within the braided structure of the cannula body 224 of the cannula 204. For example, the inner layer 234 may be arranged on the inner surface 223 of cannula body 224. In this way, the inner layer 234 defines a smooth inner surface 244 of the cannula 204 and additionally defines an inner diameter D2 of the cannula 204. In some embodiments, the value of the inner diameter D2 may range from approximately 0.09 inches (0.2286 cm) and approximately 0.29 inches (0.7366 cm).

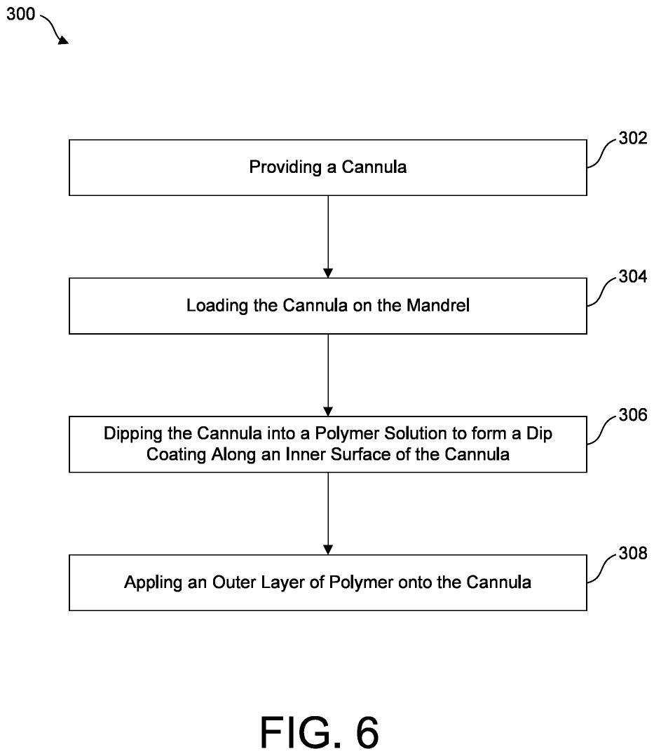

With reference now to FIG. 6, a method 300 of manufacturing cannula 204 will be described further herein. For example, at block 302, the method 300 first includes the step of providing the cannula body 224. In some embodiments, the cannula body 224 may be formed of the braided structure 238 as illustrated in FIG. 3 or may be formed of the laser cut tube 248 having the plurality of slots 242 as shown in FIG. 4. Further, in some embodiments, the cannula 204 comprises adaptors 208, shown in phantom in FIG. 3, arranged at each of the proximal portion 222 and the distal portion 216 of the cannula 204.

At block 304, the method 300 further includes loading the cannula body 224 onto a mandrel. In some embodiments, this step includes arranging the cannula body 224 onto the mandrel and using the adaptors 208 (FIG. 3) to secure the cannula body 224 onto the mandrel. At block 306, the method 300 further includes dipping the cannula body 224 and mandrel into a polymer solution to form a dip coating along the inner surface 244 of the cannula 204. More particularly, the dip coating may form the inner layer 234 which spans along the cannula 204 and is disposed on the inner surface 223 of the cannula body 224. The dip coating may also cover (e.g., partially or completely) the outer surface as well. In such arrangements, the dip coating on the outer surface may later be covered by another layer such as the outer layer 230 such that the dip coating is positioned between the outer surface and the outer layer 230. As previously described, the polymer solution, and thus the inner layer 234, may be formed of at least one of polyurethane, silicone, HYTREL®, PEBAX®, or various other thermoformed or thermoset polymers.

In the embodiments wherein the cannula body 224 is formed of the braided structure 238, and in the embodiments wherein there is not the adhesive layer 240 incorporated onto the cannula body 224, the step of dip coating the cannula body 224 may also cause the inner layer 234 to be interspersed on or within the braided structure 238 of the cannula body 224 of the cannula 204. During the step of dipping the cannula body 224 into the polymer solution, the cannula body 224 may be continuously rotated such that the polymer solution evenly coats the inner surface 223 of cannula body 224. In this way, the polymer solution does not gather and solidify unevenly around different spots on the cannula 204. More particularly, the mandrel, and thus the cannula body 224, may be rotated at a speed between approximately 5 rotations per minute (rpm) and approximately 50 rpm. In some embodiments, the method 300 may also include heat curing the inner layer 234 once the dipping of the cannula 204 into the polymer solution is completed.

At block 308, the method 300 further includes applying an outer layer of polymer onto the cannula 204. For example, this includes applying the outer layer 230 to the outer surface 225 of the cannula body 224. The applying of the outer layer 230 may include attaching, coupling, adhering, or otherwise attaching the outer layer 230 onto the outer surface 225 of the cannula body 224. In some embodiments, an adhesive layer 240 is placed around the outer surface 225 of the cannula body 224 prior to the placing of the outer layer 230 onto the cannula 204. In some embodiments, the adhesive layer 240 may be composed of DYMAX® 204-CTH, DYMAX® 203A-CTH-F, or various other applicable materials. In this way, the outer layer 230 may be secured onto the adhesive layer 240 and thus onto the cannula body 224 to increase the strength of the attachment between the outer layer 230 and the cannula body 224.

The above described steps of dip coating the cannula 204, and more particularly the cannula body 224, to form the inner layer 234 of the cannula 204 may provide several advantages. For example, the thickness of the inner layer 234 may easily be optimized through modification of the polymer solution and/or the amount of polymer solution applied to the cannula 204. Further, the dip coating process may cause the inner layer 234 to have a smooth inner diameter D1 spanning the inner surface 244 of the cannula 204. The smooth inner surface 244 of the cannula 204 reduces the chance of thrombosis or aggregate formation as the blood passes through the cannula 204. Further, coating the cannula 204 with the inner layer 234 composed of a polymer allows for a lubricious and flexible inner surface 244 of the cannula 204, increasing the ease with which that blood flows through the cannula 204.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method of coating a cannula for use with a circulatory support device, the cannula having a proximal portion opposite a distal portion and a cannula body extending between the distal portion and the proximal portion, the cannula body defined by an inner surface and an outer surface, the method comprising:
providing the cannula body;
loading the cannula body onto a mandrel;
dipping the cannula body into a polymer solution to form a dip coating along the inner surface of the cannula body; and
applying an outer layer of polymer onto the cannula.

2. The method of claim 1, wherein the cannula body is a braided structure composed of nitinol.

3. The method of claim 1, wherein the cannula body is a laser cut tube of nitinol or a coil structure composed of nitinol.

4. The method of claim 1, wherein the polymer solution is composed of a thermoset polymer.

5. The method of claim 1, wherein the outer layer of polymer applied to the cannula is a layer of a thermoset polymer.

6. The method of claim 1, wherein dipping the cannula body into the polymer solution includes rotating the mandrel and the cannula body such that the polymer solution is disposed evenly along the inner surface of the cannula body.

7. The method of claim 6, wherein the mandrel is rotated at a speed of between approximately 5 rpm and approximately 50 rpm.

8. The method of claim 1, wherein the outer layer defines an outer diameter.

9. The method of claim 8, wherein the outer diameter has a value ranging between approximately 0.1 inches and approximately 0.3 inches.

10. The method of claim 1, wherein the dip coating defines an inner layer, and the inner layer defines an inner diameter of the cannula.

11. The method of claim 10, wherein the inner diameter has a value ranging between approximately 0.09 inches and approximately 0.29 inches.

12. The method of claim 1, wherein prior to applying the outer layer onto the cannula, the method further includes attaching an adhesive layer to the outer surface of the cannula body.

13. The method of claim 12, wherein applying the outer layer onto the cannula includes attaching the outer layer to the adhesive layer.

14. A method of coating a cannula for use with a circulatory support device, the cannula having a proximal portion opposite a distal portion and a cannula body extending between the distal portion and the proximal portion, the method comprising:
providing the cannula body;
loading the cannula body onto a mandrel;
dipping the cannula body into a polymer solution to form an inner layer of coating along a surface of the cannula body;
attaching an adhesive layer onto the cannula body; and
applying an outer layer of polymer onto the cannula.

15. The method of claim 14, wherein the inner layer defines an inner diameter of the cannula and the outer layer of the polymer defines an outer diameter of the cannula.

16. The method of claim 15, wherein the inner diameter has a value of between approximately 0.09 inches and approximately 0.29 inches.

17. The method of claim 15, wherein the outer diameter has a value of between approximately 0.1 inches and approximately 0.3 inches.

18. The method of claim 14, wherein the polymer solution is composed of polyurethane or silicone.

19. The method of claim 14, wherein the polymer of the outer layer is composed of polyurethane or silicone.

20. The method of claim 14, wherein dipping the cannula body into the polymer solution includes rotating the mandrel and the cannula body such that the polymer solution is disposed evenly along an inner surface of the cannula body.
